(19) United States
(12) Patent Application Publication
(10) Pub. No.: US 2004/0151738 A1
(43) Pub. Date: Aug. 5, 2004

(54) Satiating dietetic product

(76) Inventors: Eric Oriol, Sur Marne (FR); Gilles Fromentin, Antony (FR)

Correspondence Address: SUGHRUE MION, PLLC, 2100 PENNSYLVANIA AVENUE, N.W., SUITE 800, WASHINGTON, DC, 20037, US

(21) Appl. No.: 10/478,104

(22) PCT Filed: May 21, 2002
(86) PCT No.: PCT/FR02/01700

(30) Foreign Application Priority Data
May 22, 2001 (FR) 01/06717

(51) Int. Cl. A61K 35/70

(52) U.S. Cl. 424/195.16

(57) ABSTRACT

A dietetic product belonging to the family of food products, complements or supplements for slimming or anti-obesity and appetite suppressing or satiating food products, complements or supplements containing an efficient dose of food proteins, 70% of which at least are hydrolysed yeast proteins. The invention also concerns the use of the dietetic product in an aesthetic slimming diet. The invention further concerns the use of hydrolysed yeast proteins in the preparation of such a dietetic product.

The present invention relates to a dietetic product, to applications thereof and to the preparation thereof.

It is known that correct functioning of the body of higher living beings requires, firstly, an amount of energy and, secondly, essential nutrients which it cannot synthesize, such as certain amino acids contained in proteins or vitamins. These essential nutrients must be provided by the diet.

The food proteins are preferably “good quality” proteins such as, for example, milk proteins and egg proteins. The good quality proteins are preferably proteins having a PER (Protein Efficiency Ratio) value of above 2.

It is acknowledged that hydrolyzed yeast proteins are good quality proteins.

It is known that yeast extracts rich in hydrolyzed yeast proteins are also rich in vitamins and in trace elements. Yeast extracts also provide a meat-flavor enhancing effect.

In view of their abovementioned properties, yeast extracts are used as a flavor-enhancing food preparation in cooked dishes. Yeast extracts are also used in energizing food preparations, such as meat broths and sports drinks, and also as a provider of vitamins and mineral trace elements in some dietetic products.

Document WO-A-96/38057 describes the addition of unspecified amounts of yeast extract to foodstuffs as a flavor-enhancing preparation.

Document GB-A-698864 describes nutritional preparations containing elements of the vitamin B complex, said nutritional preparations possibly containing hydrolyzed yeast proteins, but not containing an amount or dose of proteins sufficient to cause, after consumption of the nutritional preparation, an appetite-suppressing or satiating effect.

The aim of the present invention is to provide novel dietetic products belonging to the family of food products, supplements or complements (=additives) for a slimming or anti-obesity diet and appetite-suppressing or satiating food products, supplements or complements.

The invention relates, firstly, to a dietetic product and in particular a dietetic product belonging to the family of food products, supplements or complements for a slimming or anti-obesity diet and appetite-suppressing or satiating food products, supplements or complements, containing at least one yeast derivative, said dietetic product containing an effective amount or dose of food proteins, at least 70%, and preferably at least 80%, or more preferably at least 90%, of which are hydrolyzed yeast proteins.

All the percentages are percentages by weight. The protein contents are evaluated by the usual formula Ntotal*6.25 (in which Ntotal=NKjeldahl).

In the context of the present invention, the expression “slimming or anti-obesity diet” is understood to mean diets aimed at reducing or avoiding an increase in adipose material in an animal, or aimed at decreasing the adipose material present in said animal. The animal is typically a mammal and in particular a human.

In the present context, the expression “effective amount or dose of proteins” is understood to mean an amount of proteins sufficient for its consumption to cause an appetite-suppressing or satiating effect in the animal, i.e. for its consumption to temporarily cause a reduction, or even a suppression, of the desire to eat.

The present invention demonstrates, for the first time, the fact that, among good quality proteins, hydrolyzed yeast proteins, such as hydrolyzed proteins of Saccharomyces cerevisiae, have a particularly significant satiating effect or appetite-suppressing effect.

This unexpected property of hydrolyzed yeast proteins means that these proteins are particularly advantageous for slimming or anti-obesity diets.

It has in fact been noted, surprisingly, that the replacement, in a diet, of at least part of the good quality proteins, such as the milk proteins, with an proteinic equivalent amount of hydrolyzed yeast proteins leads to a reduction in the adipose material in the animal.

The term “yeast derivative” refers to any yeast derivative, such as the hydrolyzed yeast proteins and the yeast fibers. The hydrolyzed yeast proteins are preferably enzyme hydrolysates of the yeast proteins or yeast autolysates, the yeast autolysates being the result of digestion of the cellular content of the yeast cells by their own enzymes, which may or may not be supplemented by the addition of proteases, such as papain. The yeast fibers are defined as the insoluble portion of the yeast cells after autolysis of said yeast cells and/or after enzyme hydrolysis of the content of said yeast cells by proteases.

According to a first embodiment, the product according to the invention is a non pharmaceutical dietetic product for an esthetic slimming diet. According to an alternative embodiment, the product according to the invention is a medicinal product for a diet for the clinical treatment of obesity.

The dietetic product according to the invention preferably also contains food fibers, preferably yeast fibers.

The product according to the invention may, for example, contain a yeast protein enzyme hydrolysate, and in particular a yeast autolysate.

In the dietetic product according to the invention, all the hydrolyzed yeast proteins are advantageously present in the form of one or more yeast protein enzyme hydrolysates, and in particular in the form of one or more yeast autolysates.

The dietetic product according to the invention may also comprise other food ingredients, such as, for example, vitamins, minerals, trace elements, etc. The product according to the invention optionally contains other ingredients, such as flavorings and/or food dyes.

The dietetic product may be provided in several forms, inter alia in the form of a tablet, pill or gel capsule, in the form of a biscuit or of a preparation of the snack or ready-for-consumption product type, or else in the form of a drink or in the form of a powder to be dissolved or dispersed in a drink.

The product according to the invention preferably contains at least one derivative of at least one yeast belonging to the genus Saccharomyces. The product according to the invention in particular advantageously contains hydrolyzed proteins of at least one yeast belonging to this genus. According to a particular embodiment, the yeast derivative(s) present in the dietetic product are all derivatives of one or more yeasts belonging to the genus Saccharomyces.

The product according to the invention more particularly preferably contains at least one derivative of at least one yeast belonging to the species Saccharomyces cerevisiae. The product according to the invention in particular advantageously contains hydrolyzed proteins of at least one yeast belonging to this species. According to a particular embodiment, the yeast derivative(s) present in the product are all derivatives of one or more yeast belonging to the species Saccharomyces cerevisiae.

The yeast derivatives may, for example, be derivatives of bakers' yeast, derivatives of brewers' yeast and/or derivatives of a yeast which can be used for or which has been used for the production of alcohol.

The present invention also relates to the use of the product according to the invention in the treatment of an animal, for obtaining esthetic slimming, the animal being typically a mammal, and more particularly a human.

The invention also relates to the use of hydrolyzed yeast proteins as an appetite-suppressing or satiating agent in an esthetic slimming diet. The present invention relates in particular to the use, in support of an esthetic slimming diet, of a dietetic product belonging to the family of food products, supplements or complements for a slimming or anti-obesity diet and appetite-suppressing or satiating food products, supplements or complements, containing a yeast derivative, and more particularly to the use of such a dietetic product containing an effective amount or dose of food proteins, at least 70%, and preferably at least 80%, or at least 90%, of which are hydrolyzed yeast proteins.

It should be noted that the use of a dietetic product for esthetic purposes differs from a pharmaceutical or veterinary use of such a product in the clinical treatment of obesity.

The present invention also relates to the preparation of dietetic products belonging to the family of food products, supplements or complements for a slimming or anti-obesity diet and appetite-suppressing or satiating food products, supplements or complements, containing a yeast derivative, said preparation comprising the addition of hydrolyzed yeast proteins.

The present invention relates in particular to the use of hydrolyzed yeast proteins as an appetite-suppressing or satiating agent, in the preparation of a dietetic product belonging to the family of food products, supplements or complements for a slimming or anti-obesity diet and appetite-suppressing or satiating food products, supplements or complements.

The invention relates in particular to the use of an effective amount or dose of food proteins, at least 70%, and preferably at least 80%, or more preferably at least 90%, of which are hydrolyzed yeast proteins, in the preparation of such a dietetic product.

The hydrolyzed yeast proteins are preferably used in combination with food fibers, such as yeast fibers.

The hydrolyzed yeast proteins are thus advantageously used in the form of one or more yeast protein enzyme hydrolysates, preferably in the form of one or more yeast autolysates.

The yeast hydrolysates or yeast autolysates are preferably characterized by a protein degradation level, defined by the ratio of the amino nitrogen or formol nitrogen to the total nitrogen, of at least 25%, and preferably of at least 40%.

As mentioned above, the present invention originates from the surprising observation that, compared to the other known good quality proteins, hydrolyzed yeast proteins produce a slimming effect in animals, including humans.

Said surprising effect of the dietetic products according to the invention is illustrated below with reference to experimental results on rats.

Experiment

Animals

32 male Wistar rats weighing 276.4±4.2 g were housed individually in metal cages with grilles and placed in a thermostatic room at 24±1° C. with a 12 h/12 h day/night cycle.

Habituating Diet

The rats were allowed to become accustomed, for one week, to a diet, referred to as P-milk diet, containing 14% of total milk proteins and consisting of a concentrate of total milk proteins (casein+lactoserum) commonly used in specific dietetic feeding and hyperproteinated products. The proteinic content of the total milk proteins was 89%.

They were given food and water ad libitum.

In order to avoid losses, the food was given in liquid form.

Experimental Diets

After one week of the habituating diet, the rats were divided up randomly into four groups of 8 rats, referred to respectively as P-milk group, P-milk-F group, P-yeast group and P-yeast-F group.

The rats from each group were then fed at will, for 21 days, with a diet bearing the name of their group. The composition of each of the P-milk, P-milk-F, P-yeast and P-yeast-F diets is given in table 1.

The protein and solids contents and the energy values of the four diets are given in table 2.

The P-milk group, which continued to be fed with the P-milk habituating diet, plays the role of control group.

The compositions of the P-milk, P-milk-F, P-yeast and P-yeast-F diets derive from the composition of the standardized AIN93M diet (as described in the report “AIN-93 Purified Diets for Laboratory Rodents of the American Institute of Nutrition Ad Hoc writing Committee on the Reformulation of the AIN-76A Rodent Diet”).

The hydrolyzed yeast proteins were yeast extracts as sold by the company BIO-SPRINGER under the trade mark SPRINGER and having the following characteristics:

solids 96%

proteins N*6.25=74%

Namino/Ntotal ratio=50%,

the Namino being measured by the Sorensen formol nitrogen method, Ntotal being the Kjeldahl nitrogen.

The yeast fibers are yeast cell walls rich in glucans and in mannans and having the following approximative characteristics:

glucans: 24%,

mannans: 22%,

lipids: 20%,

proteins: 15%,

ash: 4%.

TABLE 1

| | | MDiet | | | |
|---|---|---|---|---|---|
| | | P- | P-milk- | P- | P-yeast- |
| | | milk | F | yeast | F |
| Ingredients (g/kg) | Total milk proteins | 140.0 | 140.0 | — | — |
| | Hydrolyzed yeast | — | — | 170.0 | 170.0 |
| | proteins | | | | |
| | Sucrose | 100.3 | 89.1 | 96.6 | 89.0 |
| | Starch | 622.4 | 582.8 | 596.6 | 552.9 |
| | AIN 93M mineral | 35.0 | 35.0 | 35.0 | 35.0 |
| | salt mixture | | | | |
| | AIN 93M vitamin | 10.0 | 10.0 | 10.0 | 10.0 |
| | mixture | | | | |
| | Soya oil | 40.0 | 40.0 | 40.0 | 40.0 |
| | Cellulose | 50.0 | — | 50.0 | — |
| | Yeast fiber | — | 100.8 | — | 100.8 |
| | Choline bitartrate | 2.3 | 2.3 | 2.3 | 2.3 |

TABLE 2

| | Diet | | | |
|---|---|---|---|---|
| | P-milk | P-milk-F | P-yeast | P-yeast-F |
| Solids content (Sc) | 90.7 | 91.2 | 87.8 | 88.3 |
| (%) | | | | |
| Proteinic content | 14 | 14 | 17 | 17 |
| (%) | | | | |
| Proteinic content | 89 | 89 | 72 | 72 |
| of the protein (%) | | | | |
| Proteinic content | 0.125 | 0.125 | 0.125 | 0.125 |
| of the solids of | | | | |
| the formulation (%) | | | | |
| Energy value (kJ/g) | 14.9 | 14.1 | 14.4 | 13.6 |

Body Composition of the Rats

At the end of the 21 days of experimental diets, the rats were made to fast on day 22. The following day, the rats were anesthetized with sodium pentobarbital and dissected. The following tissues were extracted and weighed: liver, heart, kidneys, white adipose tissues, brown adipose tissue, head, skin and carcass.

Experimental Results

a) Behavior

During the week of the habituating diet and during the 21 days of experimental diets, the animals subjected to the various diets had a normal behavior (no nervousness).

b) Weight

During the 21 days of the experimental diets, there is no significant difference between the weight of the rats in the P-milk control group and those in the P-milk-F group. After 21 days of experimental diets, the weight of the rats in the P-milk control group was 411±7 g, while the weight of the rats in the P-milk-F group was 413±11 g.

On the other hand, from the first days of the experimental diets, the weight of the rats belonging to the P-yeast and P-yeast-F groups was lower than the weight of the rats belonging to the P-milk and P-milk-F groups. The weight of the rats belonging to the P-yeast-F group was, in addition, slightly lower than the weight of the rats belonging to the P-yeast group. After 21 days of experimental diets, the weight of the rats belonging to the P-yeast group was 362±10 g. The weight of the rats belonging to the P-yeast-F group was 350±12 g.

After 21 days of experimental diets, the weight of the rats in the P-yeast group was therefore 12% less than that of the rats in the P-milk control group, while the weight of the rats in the P-yeast-F group was 15% less than that of the rats in the P-milk control group.

c) Energy Ingested

Over the 21 days of experimental diets, the daily energy ingested was highest for the rats of the P-milk control group.

Over the period of the experimental diets, the daily energy ingested by the rats in the P-milk-F group was on average 13% lower than that ingested by the rats in the P-milk control group.

The average daily energy ingested during this period by the rats in the P-yeast group was 37% lower, while the average daily energy ingested by the rats in the P-yeast-F group was 50% lower than that ingested by the rats in the P-milk control group.

d) Body Composition

Weight

The weight of the animals at the time of sacrifice, and which had therefore been fasting since the day before, is decreased by 10% in the rats of groups P-yeast (348±13 g) and P-yeast-F (353±12 g) compared to the P-milk control group (390±6 g). The weight of the rats in the P-milk-F group (405±12 g) is not significantly different from that of the rats in the P-milk control group.

Carcass

After 21 days of experimental diets, there is no significant difference between the weights of the carcasses of the rats in the four experimental groups P-milk (161±4 g), P-milk-F (165±4 g), P-yeast (149±10 g) and P-yeast-F (150±7 g).

White Adipose Tissue

After 21 days of experimental diets, the weights of the white adipose tissues of the rats in the P-milk and P-milk-F groups were not significantly different (P-milk: 65±4 g; P-milk-F: 65±7 g).

Similarly, the weights of the white adipose tissues of the rats in the P-yeast and P-yeast-F groups were not significantly different either (P-yeast: 47±3 g; P-yeast-F: 42±4 g).

On the other hand, the weights of the white adipose tissues of the rats in the P-yeast and P-yeast-F groups fed with hydrolyzed yeast proteins were clearly less than the weights of the rats in the P-milk and P-milk-F groups fed with total milk proteins. The weight of the white adipose tissues of the rats in the P-yeast group was 28% lower than the weight of the white adipose tissues of the rats in the P-milk control group. The weight of the white adipose tissues of the rats in the P-yeast-F group was 35% lower than the weight of the white adipose tissues of the rats in the P-milk control groups.

Conclusion

The experimental results show that the replacement, in a diet, of total milk proteins with an identical proteinic amount of hydrolyzed yeast proteins leads to a reduction in consumption. This satiating or appetite-suppressing effect is not associated with the diets containing hydrolyzed yeast proteins being less acceptable in terms of taste since the rats fed on diets based on hydrolyzed yeast proteins behaved in the same way as the animals fed on diets based on total milk proteins. Neither is it associated with any amino acid deficiency. In fact, although yeast proteins show a slight deficiency in sulfur-containing amino acids, the diets given to the animals had been calculated to cover their needs.

More importantly, a considerable reduction in adipose tissue is noted in the animal when total milk proteins are replaced, in a diet, with an identical proteinic amount of hydrolyzed yeast proteins.

No negative effect on the animals' health was noted subsequent to this replacement of total milk proteins.

It was noted that the apparent digestibility of the total milk proteins and of the hydrolyzed yeast proteins are virtually equivalent (respectively 90% and 88%).

Additional experiments, in which the rats fed with the diet based on total milk proteins were given an amount limited to that which the rats fed with the diet based on hydrolyzed yeast proteins had consumed the day before, showed that, in this case, the two diets produce the same result of weight change in the rats and the same effects on body composition.

The results of the experiment described in detail above (total weight 10% less, energy consumption at least 37% less, white adipose tissue weight approximately 30% less in the rats fed with the P-yeast diet based on hydrolyzed yeast proteins compared to the rats fed with the P-milk control diet based on total milk proteins) are therefore essentially the result of less ingestion by the rats fed with the diet based on hydrolyzed yeast proteins, and not of a lower food performance level of this diet.

Complementary experiments have demonstrated that this decreased consumption of the diets containing hydrolyzed yeast proteins compared to the diets containing total milk proteins was due to a greater satiating effect of the hydrolyzed yeast proteins compared to the total milk proteins. The present invention demonstrates, for the first time, a difference in satiating effect between good quality proteins.

The use of hydrolyzed yeast proteins as protein supply in a dietetic product belonging to the family of food products, supplements or complements for a slimming or anti-obesity diet and appetite-suppressing or satiating food products, supplements or complements therefore produces a significant increase in the slimming or anti-obesity effectiveness, and also in the appetite-suppressing or satiating effect, of said product.

The use according to the invention of yeast enzyme hydrolysates, and in particular of yeast autolysates, as a source of hydrolyzed yeast proteins has the additional advantage that yeast enzyme hydrolysates and yeast autolysates also constitute a source of vitamins, of mineral trace elements and of other nutrients.

1. A dietetic product belonging to the family of food products, supplements or complements for a slimming or anti-obesity diet and appetite-suppressing or satiating food products, supplements or complements, containing at least one yeast derivative, characterized in that the dietetic product contains an effective dose of food proteins, at least 70% of which are hydrolyzed yeast proteins.

2. The product as claimed in claim 1, characterized in that at least 80%, preferably at least 90%, of the food proteins are hydrolyzed yeast proteins.

3. The product as claimed in either one of the preceding claims, characterized in that it also contains yeast fibers.

4. The product as claimed in any one of the preceding claims, characterized in that it contains a yeast protein enzyme hydrolysate, preferably a yeast autolysate.

5. The product as claimed in any one of the preceding claims, characterized in that it comprises a derivative of one or more yeasts belonging to the genus Saccharomyces.

6. The product as claimed in claim 5, characterized in that it comprises a derivative of one or more yeasts belonging to the species Saccharomyces cerevisiae, and preferably of one or more of these yeasts belonging to the group of bakers' yeast, of brewers' yeast, or of yeast which can be used for or which have been used for the production of wine or of alcohol.

7. The product as claimed in any one of the preceding claims, characterized in that the product is a food product, supplement or complement for mammals, in particular for humans.

8. The use of hydrolyzed yeast proteins as an appetite-suppressing or satiating agent in an esthetic slimming diet.

9. The use, in an esthetic slimming diet, of a dietetic product belonging to the family of food products, supplements or complements for a slimming or anti-obesity diet and appetite-suppressing or satiating food products, supplements or complements, containing at least one yeast derivative, characterized in that the dietetic product contains an effective dose of food proteins, at least 70% of which are hydrolyzed yeast proteins.

10. The use as claimed in claim 9, characterized in that at least 80%, preferably at least 90%, of said food proteins are hydrolyzed yeast proteins.

11. The use as claimed in either one of claims 9 and 10, characterized in that it comprises the oral administration of the dietetic product.

12. The use of hydrolyzed yeast proteins as an appetite-suppressing or satiating agent, in the preparation of a dietetic product belonging to the family of food products, supplements or complements for a slimming or anti-obesity diet and appetite-suppressing or satiating food products, supplements or complements.

13. The use of an effective dose of food proteins, in the preparation of a dietetic product belonging to the family of food products, supplements or complements for a slimming or anti-obesity diet and appetite-suppressing or satiating food products, supplements or complements, containing at least one yeast derivative, characterized in that at least 70% of the food proteins are hydrolyzed yeast proteins.

14. The use as claimed in claim 11, characterized in that at least 80%, and preferably at least 90%, of said food proteins are hydrolyzed yeast proteins.

15. The use as claimed in any one of claims 12 to 14, characterized in that the product is a dietetic product for esthetic slimming.

16. The use as claimed in any one of claims 12 to 14, characterized in that the product is a medicinal product for the treatment of obesity.

17. The use as claimed in any one of claims 9 to 16, characterized in that the product is a food product, supplement or complement for mammals, and in particular for humans.

18. The use as claimed in any one of claims 9 to 17, characterized in that the dietetic product contains a derivative of at least one yeast belonging to the genus Saccharomyces.

19. The use as claimed in any one of claims 9 to 18, characterized in that the dietetic product contains a derivative of at least one yeast belonging to the species Saccharomyces cerevisiae, and preferably a derivative of at least one such yeast belonging to the group of bakers' yeast, brewers' yeast and yeasts which can be used for or which have been used for the production of wine or of alcohol.

20. The use as claimed in any one of claims 8 to 19, characterized in that the hydrolyzed yeast proteins are present in combination with yeast fibers.

21. The use as claimed in any one of claims 8 to 20, characterized in that the hydrolyzed yeast proteins are used in the form of a yeast protein enzyme hydrolysate, preferably in the form of a yeast autolysate.

22. The use as claimed in any one of claims 8 to 21, characterized in that the hydrolyzed yeast proteins contain, and preferably consist of, hydrolyzed proteins of a yeast belonging to the genus Saccharomyces.

23. The use as claimed in claim 22, characterized in that the hydrolyzed yeast proteins contain, and preferably consist of, hydrolyzed proteins of a yeast belonging to the species Saccharomyces cerevisiae.
